(19) United States
(12) Patent Application Publication
(10) Pub. No.: US 2025/0090078 A1
(43) Pub. Date: Mar. 20, 2025

(54) BIOLOGICAL INFORMATION ACQUIRING SYSTEM, BIOLOGICAL INFORMATION ACQUIRING APPARATUS, AND INFORMATION PROCESSING APPARATUS

(71) Applicant: SONY GROUP CORPORATION, TOKYO (JP)

(72) Inventors: RYO SASAKI, TOKYO (JP); MAO KATSUHARA, TOKYO (JP); KAZUNARI YOSHIFUJI, TOKYO (JP)

(21) Appl. No.: 18/727,022

(22) PCT Filed: Nov. 28, 2022
(86) PCT No.: PCT/JP2022/043651

§ 371 Date: Jul. 5, 2024

(30) Foreign Application Priority Data
Jan. 17, 2022 (JP) 2022-005004

(51) Int. Cl. A61B 5/374; A61B 5/00; A61B 5/291

(52) U.S. Cl. CPC A61B 5/374; A61B 5/291; A61B 5/7225

(57) ABSTRACT

To provide a biological information acquiring system, a biological information acquiring apparatus, and an information processing apparatus that make it possible to estimate biological information with a high degree of accuracy. A biological information acquiring system according to the present technology includes a plurality of potential measurement sections and a second signal processor. Each of the plurality of potential measurement sections includes a reference electrode, a measurement electrode, a biological signal generator, an analog-digital converter, and a first signal processor. The reference electrode is arranged on a measurement-target living body. The measurement electrode is arranged on the living body. The biological signal generator generates a biological signal from a difference between a potential of the reference electrode and a potential of the measurement electrode. The analog-digital converter digitally converts the biological signal. The first signal processor calculates a first feature amount using the digitally converted biological signal. The second signal processor calculates a second feature amount used to estimate a state of the living body, using the first feature amounts respectively calculated by the first signal processors respectively included in the plurality of potential measurement sections.

### TECHNICAL FIELD

The present technology relates to a biological information acquiring system, a biological information acquiring apparatus, and an information processing apparatus.

### BACKGROUND ART

A brain wave measuring apparatus that measures, for example, brain waves as biological information is typically arranged at a position close to a brain activity site, and has a configuration in which a measurement electrode, a reference electrode, and a signal processor are connected to each other through wiring, the measurement electrode detecting a biological signal, the reference electrode detecting a reference biological signal in a site such as an earlobe that exhibits less propagation of brain waves, the reference biological signal being used as a reference for the biological signal detected by the measurement electrode. In order to observe brain waves with a high signal level and estimate a biological state with a high degree of accuracy, it is necessary that a reference electrode and a detection electrode be sufficiently spaced from each other and that the reference electrode be arranged at a limited location. This results in restrictions on making an electroencephalograph smaller in size.

In recent years, efforts to acquire brain waves in a limited space such as in an ear or around an ear have attracted attention, in order to improve usability by making an apparatus smaller in size. A brain wave measuring apparatus that is attachable around an ear has been proposed, where a reference electrode is arranged on an earlobe, and a measurement electrode is arranged in an ear canal or on a portion, on the side of the head, that is situated around the ear (for example, Patent Literature 1).

### CITATION LIST

### Patent Literature

Patent Literature 1: Japanese Patent Application Laid-open No. 2018-186934

### DISCLOSURE OF INVENTION

### Technical Problem

There is a need to estimate biological information with a high degree of accuracy upon acquiring biological information using biological signals such as brain waves.

In view of the circumstances described above, it is an object of the present technology to provide a biological information acquiring system, a biological information acquiring apparatus, and an information processing apparatus that make it possible to estimate biological information with a high degree of accuracy.

### Solution to Problem

In order to achieve the object described above, a biological information acquiring system according to the present technology includes a plurality of potential measurement sections and a second signal processor.

Each of the plurality of potential measurement sections includes a reference electrode, a measurement electrode, a biological signal generator, an analog-digital converter, and a first signal processor.

The reference electrode is arranged on a measurement-target living body.

The measurement electrode is arranged on the living body.

The biological signal generator generates a biological signal from a difference between a potential of the reference electrode and a potential of the measurement electrode.

The analog-digital converter digitally converts the biological signal.

The first signal processor calculates a first feature amount using the digitally converted biological signal.

The second signal processor calculates a second feature amount used to estimate a state of the living body, using the first feature amounts respectively calculated by the first signal processors respectively included in the plurality of potential measurement sections.

According to such a configuration, a second feature amount used to estimate a state of a living body is calculated using first feature amounts respectively obtained from a plurality of potential measurement sections of which reference electrodes are situated at different positions. This makes it possible to improve the accuracy in estimating the state of the living body.

The biological information acquiring system may further include a state estimator that estimates the state of the living body on the basis of the second feature amount.

The first signal processor included in each of the plurality of potential measurement sections may calculate a power for each frequency from the digitally converted biological signal, and may normalize the power for each frequency using an integrated intensity in a specified frequency interval to calculate the first feature amount for a normalized intensity.

The second signal processor may calculate the second feature amount by performing signal averaging on the first feature amounts respectively calculated by the plurality of potential measurement sections.

The biological information acquiring system may include the plurality of potential measurement sections of which the number is at least three; from among the first feature amounts respectively calculated by the plurality of potential measurement sections, the second signal processor may set the first feature amount exhibiting a smallest value to be a reference feature amount; and the second signal processor may calculate the second feature amount by performing signal averaging on the reference feature amount and each of the first feature amounts respectively calculated by the potential measurement sections other than the potential measurement section calculating the first feature amount set to be the reference feature amount.

From among the first feature amounts respectively calculated by the plurality of potential measurement sections, the second signal processor may set the first feature amount exhibiting a smallest value to be a reference feature amount; and the second signal processor may calculate the second feature amount by subtracting the reference feature amount from each of the first feature amounts respectively calculated by the potential measurement sections other than the potential measurement section calculating the first feature amount set to be the reference feature amount.

The first signal processor included in each of the plurality of potential measurement sections may calculate, as the first feature amount, a power for each frequency from the digitally converted biological signal; the second signal processor may calculate an intermediate feature amount by performing signal averaging on the powers for each frequency that are respectively calculated by the plurality of potential measurement sections; and the second signal processor may normalize the intermediate feature amount using an integrated intensity in a specified frequency interval to calculate the second feature amount for a normalized intensity.

Each of the plurality of potential measurement sections may further include a bias electrode.

In order to achieve the object described above, a biological information acquiring apparatus according to the present technology includes a plurality of potential measurement sections.

Each of the plurality of potential measurement sections includes a reference electrode, a measurement electrode, a biological signal generator, an analog-digital converter, and a first signal processor.

The reference electrode is arranged on a measurement-target living body.

The measurement electrode is arranged on the living body.

The biological signal generator generates a biological signal from a difference between a potential of the reference electrode and a potential of the measurement electrode.

The analog-digital converter digitally converts the biological signal.

The first signal processor calculates a first feature amount using the digitally converted biological signal.

Each of the plurality of potential measurement sections may further include a communication section that transmits the first feature amount to an external apparatus.

The biological information acquiring apparatus may further include a second signal processor that calculates a second feature amount used to estimate a state of the living body, using the first feature amounts respectively calculated by the first signal processors respectively included in the plurality of potential measurement sections.

The biological information acquiring apparatus may further include a state estimator that estimates the state of the living body on the basis of the second feature amount.

In order to achieve the object described above, an information processing apparatus according to the present technology includes a second signal processor.

The second signal processor calculates a second feature amount used to estimate a state of a living body, using first feature amounts respectively calculated by first signal processors that are respectively included in a plurality of potential measurement sections.

Each of the plurality of potential measurement sections includes a reference electrode that is arranged on the living body corresponding to a measurement target; a measurement electrode that is arranged on the living body; a biological signal generator that generates a biological signal from a difference between a potential of the reference electrode and a potential of the measurement electrode; an analog-digital converter that digitally converts the biological signal; and the first signal processor calculating the first feature amount using the digitally converted biological signal.

### BRIEF DESCRIPTION OF DRAWINGS

FIG. 1 schematically illustrates a biological information acquiring system according to first to fourth embodiments of the present technology.

FIG. 2 schematically illustrates the biological information acquiring system described above.

FIG. 3 illustrates a flow of signal processing performed by the biological information acquiring system according to the first embodiment.

FIG. 4 illustrates a flow of signal processing performed by the biological information acquiring system according to the second embodiment.

FIG. 5 illustrates a flow of signal processing performed by the biological information acquiring system according to the third embodiment.

FIG. 6 illustrates a flow of signal processing performed by the biological information acquiring system according to the fourth embodiment.

FIG. 7 schematically illustrates a biological information acquiring system according to a fifth embodiment.

FIG. 8 schematically illustrates a biological information acquiring system according to a sixth embodiment.

FIG. 9 schematically illustrates a biological information acquiring system according to a seventh embodiment.

FIG. 10 schematically illustrates a biological information acquiring system according to an eighth embodiment.

FIG. 11 schematically illustrates a biological information acquiring system according to a ninth embodiment.

FIG. 12 schematically illustrates a biological information acquiring system according to a tenth embodiment.

### MODE(S) FOR CARRYING OUT THE INVENTION

Biological information acquiring systems according to embodiments of the present technology are described. In the biological information acquiring systems according to the embodiments described below, a second feature amount used to estimate a human state is calculated using a result of measuring human brain waves, where a human is an example of a measurement-target living body.

Note that an example in which brain waves are used as biological signals in order to estimate a human state is described here. However, other biological signals such as an electro-cardio potential, a myoelectric potential, and pulse waves may be used.

The respective embodiments are described below.

### First Embodiment

### [Configuration of Biological Information Acquiring System]

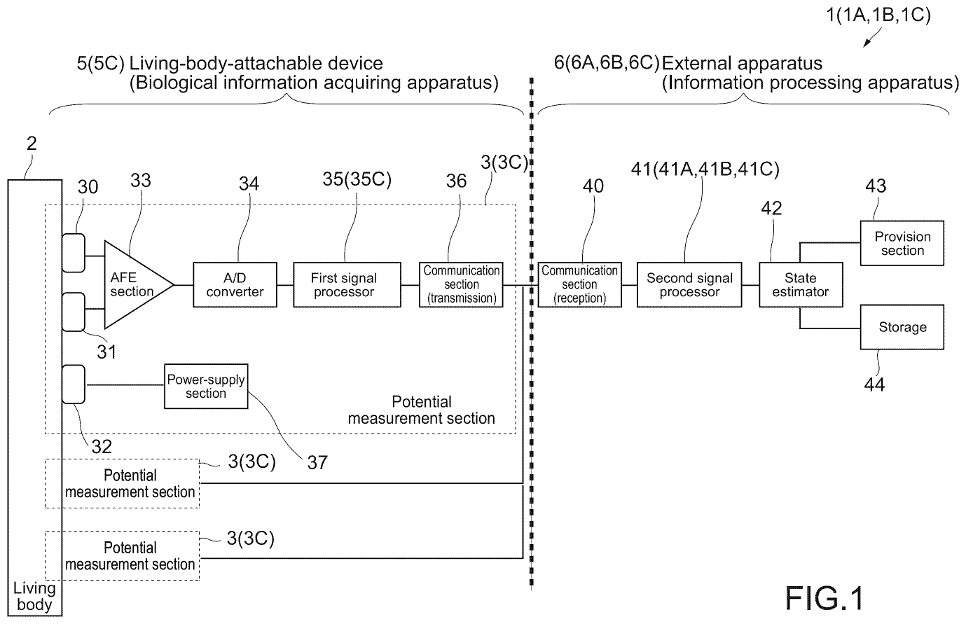

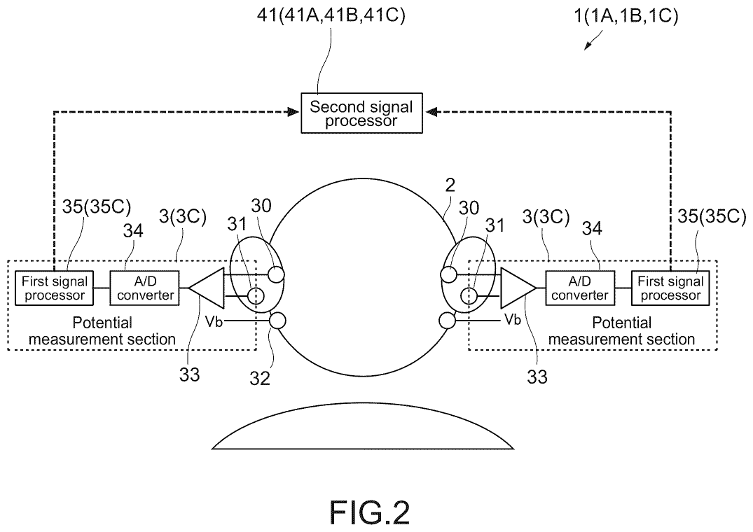

FIGS. 1 and 2 each schematically illustrate a configuration of a biological information acquiring system 1 according to the present embodiment.

As illustrated in FIG. 1, the biological information acquiring system 1 includes a living-body-attachable device 5 that serves as a biological information acquiring apparatus, and an external apparatus 6 that serves as an information processing apparatus.

The living-body-attachable device 5 is attachable to a head of a target-measurement human (a living body) 2, more particularly an ear of the human 2. The living-body-attachable device 5 includes a plurality of potential measurement sections 3. Each potential measurement section 3 includes a first signal processor 35 described later.

The external apparatus 6 is an apparatus that is different from the living-body-attachable device 5. Examples of the external apparatus 6 include other terminals such as a smartphone and a personal computer (PC), and a server apparatus and a cloud that are in a network. The external apparatus 6 includes a second signal processor 41 described later.

In the biological information acquiring system 1, a second feature amount used to estimate a state of the human 2 is calculated using brain-wave signals (biological signals) respectively acquired by the plurality of potential measurement sections 3.

### [Configuration of Living-Body-Attachable Device (Biological Information Acquiring Apparatus)]

As illustrated in FIGS. 1 and 2, the living-body-attachable device 5 includes the plurality of potential measurement sections 3. The potential measurement sections 3 have the same configuration.

For convenience, FIG. 1 illustrates the living-body-attachable device 5 including three potential measurement sections 3, and FIG. 2 illustrates the living-body-attachable device 5 including two potential measurement sections 3. However, the number of potential measurement sections 3 is not limited thereto, and it is sufficient if at least two potential measurement sections 3 are provided.

Each of the plurality of potential measurement sections 3 is a unit that includes a measurement electrode 30, a reference electrode 31, a bias electrode 32, an analog front-end section (an AFE section) 33 that serves as a biological signal generator, an analog-digital converter (an A/D converter) 34, a first signal processor 35, a communication section 36, and a power-supply section 37.

Each potential measurement section may include a plurality of measurement electrodes. An example in which the potential measurement section includes a plurality of measurement electrodes will be described in a tenth embodiment described later.

As illustrated in FIG. 2, the measurement electrode 30, the reference electrode 31, and the bias electrode 32 are arranged, for example, in or near the ear of the human 2. In the example illustrated in FIG. 2, the potential measurement section 3 of the living-body-attachable device 5 is arranged on each of right and left ears of the human 2, and brain-wave information regarding brain waves between the right and left ears can be acquired.

Typically, the measurement electrode 30 is arranged in an ear.

Typically, the reference electrode 31 is arranged on a site such as an earlobe that exhibits less propagation of brain waves.

For example, the bias electrode 32 is arranged at a position, on an earlobe, that is different from the position at which the reference electrode 31 is arranged. The bias electrode 32 matches a potential of the human 2 and a potential of a circuit. A bias electrode is provided more favorably in order to acquire highly accurate biological information.

From a difference between a potential of the reference electrode 31 and a potential of the measurement electrode 30, the analog front-end section 33 generates a brain-wave signal that is a biological signal.

The analog-digital converter 34 digitally converts the brain-wave signal (the biological signal).

The first signal processor 35 calculates a first feature amount using the digitally converted brain-wave signal.

Signal processing approaches such as frequency analysis using fast Fourier transform (FFT), wavelet analysis, empirical mode decomposition, principal component analysis (PCA), and independent component analysis (ICA) are used as methods for calculating a first feature amount, but the calculation method is not limited thereto.

An example in which the first signal processor 35 calculates a first feature amount using power that is calculated for each frequency from a brain-wave signal using frequency analysis by use of FFT, is described here.

The first signal processor 35 calculates power for each frequency from a digitally converted brain-wave signal. Further, the first signal processor 35 normalizes the power for each frequency using an integrated intensity in a specified frequency interval to calculate a first feature amount (a normalization feature amount) for a normalized intensity.

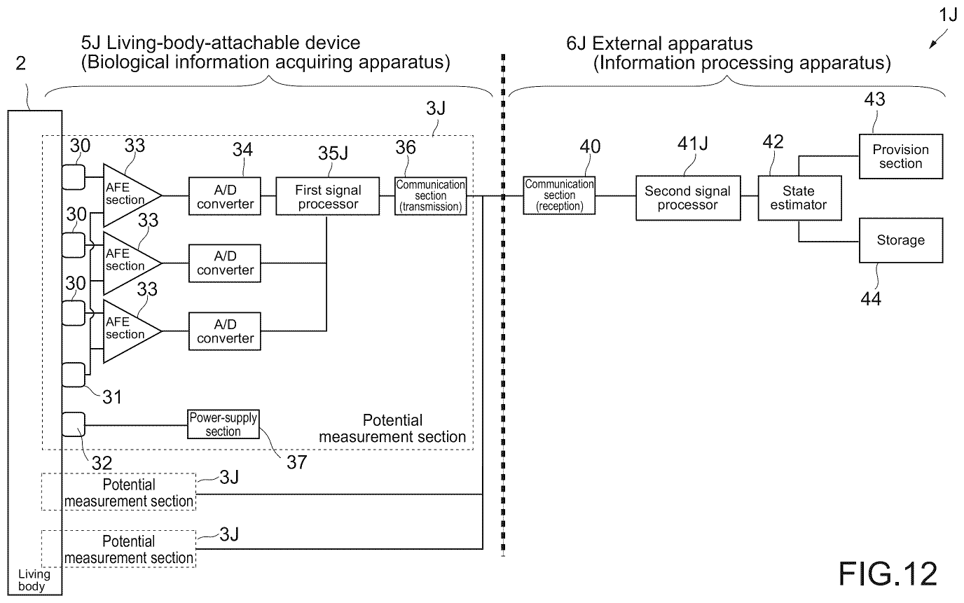

Note that, when a first feature amount is calculated using PCA or ICA, each potential measurement section has to acquire a plurality of measurement signals. Thus, each potential measurement section includes a plurality of measurement electrodes, as illustrated in FIG. 12. Such a configuration will be described later as the tenth embodiment with reference to FIG. 12. Further, each potential measurement section may also include a plurality of measurement electrodes when a calculation method using, for example, FFT that is other than PCA and ICA is used.

The communication section 36 communicates with the external apparatus 6. In the present embodiment, the communication section 36 transmits, to the external apparatus 6, a first feature amount calculated by the first signal processor 35. The communication section 36 and a communication section 40 described later may perform communication by wire or wirelessly, but favorably perform wireless communication from a usability perspective.

The power-supply section 37 supplies power to each structural element of the living-body-attachable device 5. The power-supply section 37 includes, for example, a primary battery or a secondary battery, and supplies each structural element of the living-body-attachable device 5 with power from the battery.

### [Configuration of External Apparatus (Information Processing Apparatus)]

As illustrated in FIG. 1, the external apparatus 6 includes the communication section 40, the second signal processor 41, a state estimator 42, a provision section 43, and a storage 44.

The communication section 40 communicates with the living-body-attachable device 5. In the present embodiment, the communication section 40 receives a first feature amount that is calculated by each potential measurement section 3 and transmitted from the living-body-attachable device 5.

The second signal processor 41 calculates a second feature amount used to estimate a state of the human 2, using first feature amounts respectively calculated by the first signal processors 35 respectively included in the plurality of potential measurement sections 3.

Specifically, the second signal processor 41 calculates the second feature amount by performing signal averaging on the first feature amounts respectively calculated by the plurality of potential measurement sections 3.

The state estimator 42 estimates a state of the human 2 on the basis of a second feature amount. For example, the second feature amount and state information regarding a state of a living body are stored in a state of being associated with each other in advance, and the state estimator 42 can estimate the state of the human 2 by referring to the associated information.

State information regarding the estimated state of the human 2 is transmitted to the provision section 43 and the storage 44.

The provision section 43 provides the state information regarding the estimated state of the human 2 to, for example, the measurement-target human (a user) 2 and a brain-wave measurer using sound or an image. When the information is provided using an image, the provision section 43 is, for example, a liquid crystal display or an electroluminescence (EL) display. When the information is provided using sound, the provision section 43 is, for example, a speaker.

The storage 44 stores therein, for example, the state information regarding the estimated state of the human 2, and the first and second feature amounts used to estimate the state information.

### [Flow of Processing Performed by Biological Information Acquiring System]

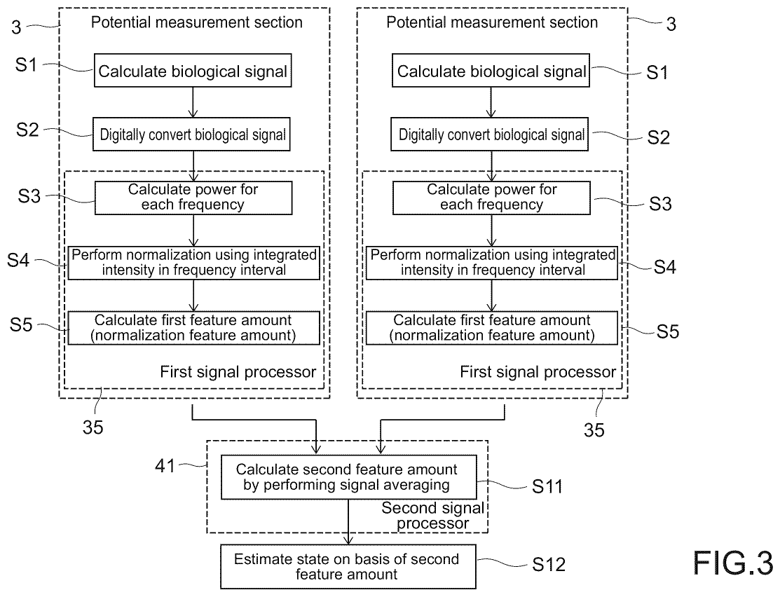

A flow of processing performed by the biological information acquiring system 1 described above is described with reference to FIG. 3.

In each potential measurement section 3 of the living-body-attachable device 5, the potential of the measurement electrode 30 and the potential of the reference electrode 31 are measured, and the analog front-end section 33 generates (S1) a brain-wave signal from a difference between the potential of the measurement electrode 30 and the potential of the reference electrode 31, as illustrated in FIG. 3.

Next, the analog-digital converter 34 digitally converts (S2) the brain-wave signal.

Next, the first signal processor 35 calculates (S3) power for each frequency from the digitally converted brain-wave signal. For example, Fourier transform or power spectrum density can be used to calculate power for each frequency.

Next, the first signal processor 35 normalizes (S4) the calculated power for each frequency (a power distribution) using an integrated intensity in a specified frequency interval to calculate (S5) a first feature amount for a normalized intensity.

The first feature amount calculated by each potential measurement section 3 is transmitted to the external apparatus 6 through the communication section 36.

As described above, information output from the living-body-attachable device 5 to the external apparatus 6 is digitized information.

The external apparatus 6 receives, through the communication section 40, the first feature amount calculated by each potential measurement section 3.

The second signal processor 41 of the external apparatus 6 calculates (S11) a second feature amount by performing signal averaging on the received first feature amounts.

Next, the state estimator 42 of the external apparatus 6 estimates (S12) a state of the human 2 on the basis of the second feature amount.

The “specified frequency interval” in S4 described above refers to a range of, for example, from 0.5 Hz to 30 Hz for typical brain waves. The specified frequency interval differs depending on a measurement target (that corresponds to brain waves in the present embodiment).

The “feature amount (a first feature amount) for a normalized intensity” in S5 described above refers to a feature amount in a checking-target frequency band (a feature amount in a frequency band to be observed upon state estimation. When, for example, an alpha wave is a checking target, a feature amount (a first feature amount) for a normalized intensity corresponds to an integrated intensity in a range of from 8 Hz to 13 Hz, which is a frequency band for the alpha wave. The alpha wave is a component that normally increases when people get relaxed.

In S11, a second feature amount can be calculated using first feature amounts respectively calculated by a plurality of different potential measurement sections 3, and, in S12, a relaxing degree of the human 2 can be estimated as a state of the human 2 on the basis of the second feature amount.

As described above, in the biological information acquiring system 1 according to the present embodiment, a brain-wave signal (a biological signal) that is acquired by each of the plurality of potential measurement sections 3 of the living-body-attachable device 5 is digitized to be converted into power for each frequency, and is transmitted to the external apparatus 6. More specifically, the first signal processor 35 of the potential measurement section 3 normalizes a brain-wave signal digitized and converted into power for each frequency, and calculates a first feature amount (a normalization feature amount). Then, the calculated first feature amount is transmitted to the external apparatus 6.

Then, the second signal processor 41 of the external apparatus 6 calculates a second feature amount used to estimate a state of the human 2, using pieces of digitized biological information (the first feature amounts) respectively acquired by a plurality of potential measurement sections 3 of which reference electrodes are situated at different positions.

Such a configuration makes it possible to increase an S/N ratio by performing normalization processing even if a level (an intensity) of a brain-wave signal acquired by each potential measurement section 3 is low. Further, a second feature amount is calculated by combining pieces of information respectively acquired by a plurality of potential measurement sections 3 of which reference electrodes are situated at different positions. This makes it possible to estimate a state of the human 2 with a high degree of accuracy.

Here, generally, a level of the acquired brain-wave signal can be increased by making a distance between the measurement electrode 30 and the reference electrode 31 sufficiently large.

In the biological information acquiring system 1 according to the present embodiment, a state of the human 2 can be estimated with a high degree of accuracy even if a level of a brain-wave signal acquired by each potential measurement section 3 of the living-body-attachable device 5 is low, as described above. Thus, in the biological information acquiring system 1, a degree of freedom in design such as arrangement positions of electrodes such as the measurement electrode 30 and the reference electrode 31 in the living-body-attachable device 5, can be increased. In other words, the measurement electrode 30 and the reference electrode 31 can be arranged such that a distance between the measurement electrode 30 and the reference electrode 31 is made smaller, while a human state can be estimated with a high degree of accuracy. This makes it possible to make the living-body-attachable device 5 smaller in size, and thus to improve the usability. Further, the increase in the degree of freedom in design makes it possible to ease constraints upon attachment, and to design a living-body-attachable device suitable for measurement as appropriate by, for example, making the device smaller in size, such that burdens imposed on a measurement target (a user) when the living-body-attachable device is attached to the measurement target, are reduced.

As described above, the present embodiment makes it possible to perform a state estimation with a high degree of accuracy and to improve the usability at the same time.

Further, each potential measurement section 3 of the living-body-attachable device 5 digitally communicates with the external apparatus 6. Consequently, the potential measurement sections 3 each being an individual unit can be made separate from each other, and the usability can be improved.

Further, each potential measurement section 3 of the living-body-attachable device 5 may communicate with the external apparatus 6 wirelessly. This makes it possible to omit analog wires between the potential measurement sections 3 of the plurality of potential measurement sections 3 each being a unit, and an analog wire between each potential measurement section 3 and the external apparatus 6.

This results in low noise due to a reduction in the number of wires. This makes it possible to perform a state estimation with a higher degree of accuracy and to improve the usability at the same time. Consequently, burdens imposed on a user when the living-body-attachable device is attached to the user can be reduced.

Here, in the case of typical brain wave measuring apparatuses, one reference electrode is arranged on an earlobe, and a plurality of measurement electrodes is arranged for the one reference electrode. This results in there being a need for wire connections corresponding to the number of the electrodes, and thus in difficulty in achieving the usability. Further, a large number of connection wires result in various kinds of noise such as environment noise and noise caused upon body movement, and this is one reason for a reduction in signal quality. Further, when, for improvement of the usability, a reference electrode and a measurement electrode are arranged closely to each other to obtain a brain wave measuring apparatus with as few wires as possible, this results in a low level of an acquirable brain-wave signal, and thus in a reduction in the accuracy in a state estimation performed using brain waves.

On the other hand, the present embodiment makes it possible to perform a state estimation with a high degree of accuracy and to improve the usability at the same time, as described above. In addition, wireless communication makes it possible to perform a state estimation with a higher degree of accuracy and to improve the usability at the same time.

In the present embodiment, the first signal processor 35 and the second signal processor 41 perform the following processing in order to calculate a second feature amount used to estimate a state of a living body.

In other words, the first signal processor 35 of the potential measurement section 3 calculates a first feature amount using a digitally converted brain-wave signal. More specifically, the first signal processor 35 calculates power for each frequency from the digitally converted brain-wave signal. Thereafter, the first signal processor 35 performs processing of normalizing the power using an integrated intensity in a specified frequency interval to calculate a first feature amount for a normalized intensity.

The second signal processor 41 of the external apparatus 6 calculates a second feature amount by performing signal averaging on the first feature amounts respectively calculated by the plurality of potential measurement sections 3.

A method for calculating a second feature amount is not limited to the method described above. Other calculation methods will be described in second to fourth embodiments. Which calculation method is to be used to calculate a second feature amount is set as appropriate according to, for example, a checking target or a signal intensity (a signal level) of an acquirable brain-wave signal, such that a state estimation can be performed with a higher degree of accuracy.

In the second and third embodiments, the description is made focused on a point different from that of the first embodiment. A structural element that is similar to the structural element described in the first embodiment is denoted by a reference numeral similar to the reference numeral used in the first embodiment, and a description thereof may be omitted. Further, also with respect to a description of processing flow, a step similar to the step described in the first embodiment is denoted in principle by a step number similar to the step number used in the first embodiment.

In a biological information acquiring system according to each of the second and third embodiments, the first signal processor included in each of the plurality of potential measurement sections calculates a first feature amount using a digitally converted brain-wave signal, and the second signal processor calculates a second feature amount used to estimate a state of a human, using the first feature amounts calculated by the respective potential measurement sections, as in the case of the first embodiment.

An example in which, in order to calculate a first feature amount, power is calculated for each frequency as the first feature amount using frequency analysis by use of FFT is described in the second and third embodiments, as in the case of the first embodiment. However, a first feature amount may be calculated using one of wavelet analysis, empirical mode decomposition, PCA, and ICA.

The respective embodiments are described below.

### Second Embodiment

### [Configuration of Biological Information Acquiring System]

The present embodiment is primarily different from the first embodiment in that the second signal processor performs different signal processing.

FIGS. 1 and 2 each schematically illustrate a configuration of a biological information acquiring system 1A according to the present embodiment.

As illustrated in FIG. 1, the biological information acquiring system 1A includes the living-body-attachable device 5 serving as a biological information acquiring apparatus, and an external apparatus 6A that serves as an information processing apparatus.

The living-body-attachable device 5 includes a plurality of potential measurement sections 3. Each potential measurement section 3 includes the first signal processor 35.

The external apparatus 6A is an apparatus that is different from the living-body-attachable device 5. Examples of the external apparatus 6A include other terminals such as a smartphone and a personal computer (PC), and a server apparatus and a cloud that are in a network. The external apparatus 6A includes a second signal processor 41A described later.

### [Configuration of Living-Body-Attachable Device (Biological Information Acquiring Apparatus)]

The living-body-attachable device 5 has a configuration similar to the living-body-attachable device 5 according to the first embodiment.

As illustrated in FIGS. 1 and 2, the living-body-attachable device 5 includes the plurality of potential measurement sections 3. The potential measurement sections 3 have the same configuration. In the present embodiment, at least three potential measurement sections 3 are provided.

Each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates power for each frequency from a digitally converted brain-wave signal, and normalizes the power for each frequency using an integrated intensity in a specified frequency interval to calculate a first feature amount (a normalization feature amount) for a normalized intensity.

The communication section 36 communicates with the external apparatus 6A. In the present embodiment, the communication section 36 transmits, to the external apparatus 6A, a first feature amount calculated by the first signal processor 35.

### [Configuration of External Apparatus (Information Processing Apparatus)]

As illustrated in FIG. 1, the external apparatus 6A includes the communication section 40, the second signal processor 41A, the state estimator 42, the provision section 43, and the storage 44.

The communication section 40 communicates with the living-body-attachable device 5. The communication section 40 receives a first feature amount that is calculated by each potential measurement section 3 and transmitted from the living-body-attachable device 5.

The second signal processor 41A calculates second feature amounts used to estimate a state of the human 2, using first feature amounts respectively calculated by the first signal processors 35 respectively included in the plurality of potential measurement sections 3.

Specifically, the second signal processor 41A compares magnitudes of first feature amounts respectively calculated by at least three potential measurement sections 3, and sets the first feature amount exhibiting a smallest value to be a reference feature amount.

Next, the second signal processor 41A calculates a second feature amount by performing signal averaging on the reference feature amount and each of the first feature amounts respectively calculated by the potential measurement sections 3 other than the potential measurement section 3 calculating the first feature amount set to be the reference feature amount.

For example, it is assumed that three potential measurement sections are a first potential measurement section, a second potential measurement section, and a third potential measurement section, and that a first feature amount calculated by the second potential measurement section is set to be a reference feature amount. The second signal processor 41A calculates a second feature amount by performing signal averaging on the reference feature amount and the first feature amount for each of the first potential measurement section and the third potential measurement section. In other words, two second feature amounts that are a second feature amount obtained by performing signal averaging on the reference feature amount and the first feature amount calculated by the first potential measurement section, and a second feature amount obtained by performing signal averaging on the reference feature amount and the first feature amount calculated by the third potential measurement section, are calculated.

The comparison between first feature amounts (normalization feature amounts) may be set in advance with respect to hardware, or first feature amounts may be compared automatically using an amplitude of a brain-wave signal or using an integrated intensity (for example, an integrated intensity in a range of from 8 Hz to 13 Hz when the alpha wave is a target) in a frequency band, for a feature amount, that is to be observed upon state estimation.

The state estimator 42 estimates a state of the human 2 on the basis of the second feature amounts calculated by the second signal processor 41A. State information regarding the estimated state of the human 2 is transmitted to the provision section 43 and the storage 44.

### [Flow of Processing Performed by Biological Information Acquiring System]

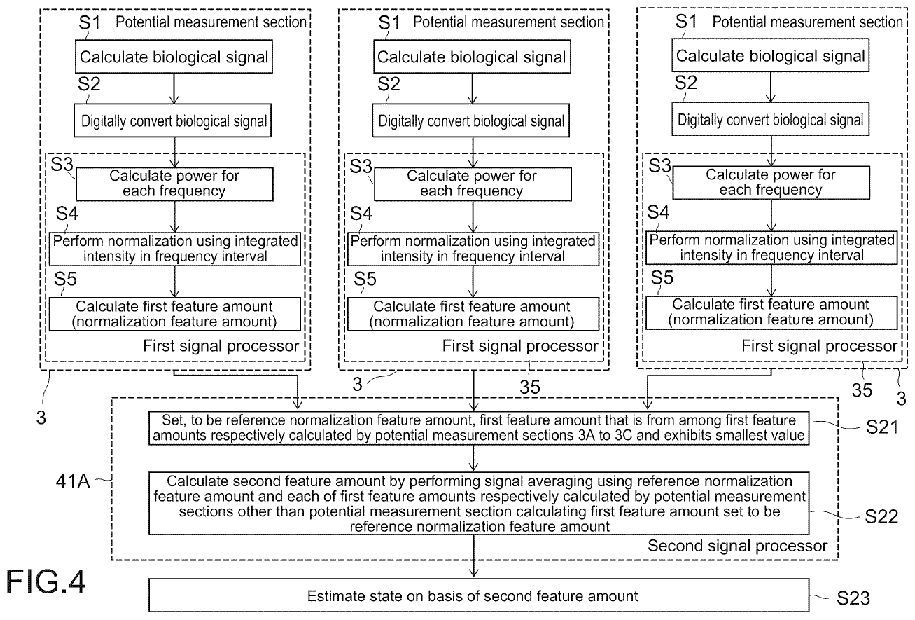

A flow of processing performed by the biological information acquiring system 1A described above is described with reference to FIG. 4.

In each potential measurement section 3 of the living-body-attachable device 5, the potential of the measurement electrode 30 and the potential of the reference electrode 31 are measured, and the analog front-end section 33 generates (S1) a brain-wave signal from a difference between the potential of the measurement electrode 30 and the potential of the reference electrode 31, as illustrated in FIG. 4.

Next, the analog-digital converter 34 digitally converts (S2) the brain-wave signal.

Next, the first signal processor 35 calculates (S3) power for each frequency from the digitally converted brain-wave signal.

Next, the first signal processor 35 normalizes (S4) the calculated power for each frequency (a power distribution) using an integrated intensity in a specified frequency interval to calculate (S5) a first feature amount for a normalized intensity.

The first feature amount calculated by each potential measurement section 3 is transmitted to the external apparatus 6A through the communication section 36.

As described above, information output from the living-body-attachable device 5 to the external apparatus 6A is digitized information.

The external apparatus 6A receives, through the communication section 40, the first feature amount calculated by each potential measurement section 3.

The second signal processor 41A of the external apparatus 6A compares magnitudes of the first feature amounts respectively calculated by the plurality of potential measurement sections 3, and sets the first feature amount exhibiting a smallest value to be a reference feature amount (S21).

Next, the second signal processor 41A calculates a second feature amount by performing signal averaging on the reference feature amount and each of the first feature amounts respectively calculated by the potential measurement sections 3 other than the potential measurement section 3 calculating the first feature amount set to be the reference feature amount (S22).

Next, the state estimator 42 of the external apparatus 6A estimates a state of the human 2 on the basis of the second feature amounts (S23).

As described above, in the biological information acquiring system 1A according to the present embodiment, a brain-wave signal (a biological signal) that is acquired by each of the plurality of potential measurement sections 3 of the living-body-attachable device 5 is digitized to be converted into power for each frequency. Then, the power is normalized to calculate a first feature amount (a normalization feature amount), and the calculated first feature amount is transmitted to the external apparatus 6A.

Then, the second signal processor 41A of the external apparatus 6A calculates second feature amounts used to estimate a state of the human 2, using pieces of digitized biological information (the first feature amounts) respectively acquired by a plurality of potential measurement sections 3 of which reference electrodes are situated at different positions.

As in the case of the first embodiment, such a configuration makes it possible to increase an S/N ratio by performing normalization processing even if a level (an intensity) of a brain-wave signal acquired by each potential measurement section 3 is low. Further, second feature amounts are calculated by combining pieces of information respectively acquired by a plurality of potential measurement sections 3 of which reference electrodes are situated at different positions. This makes it possible to estimate a state of the human 2 with a high degree of accuracy.

Further, each potential measurement section 3 of the living-body-attachable device 5 digitally communicates with the external apparatus 6A. Consequently, the potential measurement sections 3 each being an individual unit can be made separate from each other, and the usability can be improved.

Further, a degree of freedom in design of the living-body-attachable device 5 can be increased. This makes it possible to make the living-body-attachable device 5 smaller in size, and thus to improve the usability. Further, the increase in the degree of freedom in design makes it possible to design a living-body-attachable device suitable for measurement as appropriate by, for example, making the device smaller in size, such that burdens imposed on a measurement target when the living-body-attachable device is attached to the measurement target, are reduced.

Furthermore, when a feature amount exhibiting a large change is compared with a first feature amount exhibiting a smallest change using the first feature amount exhibiting a smallest change as a reference, as in the case of the present embodiment, this makes it possible to estimate a state of a living body with a higher degree of accuracy.

### Third Embodiment

### [Configuration of Biological Information Acquiring System]

The present embodiment is primarily different from the first embodiment in that the second signal processor performs different signal processing.

FIGS. 1 and 2 each schematically illustrate a configuration of a biological information acquiring system 1B according to the present embodiment.

As illustrated in FIG. 1, the biological information acquiring system 1B according to the present embodiment includes the living-body-attachable device 5 serving as a biological information acquiring apparatus, and an external apparatus 6B that serves as an information processing apparatus.

The living-body-attachable device 5 includes a plurality of potential measurement sections 3. Each potential measurement section 3 includes the first signal processor 35.

The external apparatus 6B is an apparatus that is different from the living-body-attachable device 5. Examples of the external apparatus 6B include other terminals such as a smartphone and a personal computer (PC), and a server apparatus and a cloud that are in a network. The external apparatus 6B includes a second signal processor 41B described later.

### [Configuration of Living-Body-Attachable Device (Biological Information Acquiring Apparatus)]

The living-body-attachable device 5 has a configuration similar to the living-body-attachable device 5 according to the first embodiment.

As illustrated in FIGS. 1 and 2, the living-body-attachable device 5 includes the plurality of potential measurement sections 3. The potential measurement sections 3 have the same configuration.

Each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates power for each frequency from a digitally converted brain-wave signal, and normalizes the power for each frequency using an integrated intensity in a specified frequency interval to calculate a first feature amount (a normalization feature amount) for a normalized intensity.

The communication section 36 communicates with the external apparatus 6. In the present embodiment, the communication section 36 transmits, to the external apparatus 6B, a first feature amount calculated by the first signal processor 35.

### [Configuration of External Apparatus (Information Processing Apparatus)]

As illustrated in FIG. 1, the external apparatus 6B includes the communication section 40, the second signal processor 41B, the state estimator 42, the provision section 43, and the storage 44.

The communication section 40 communicates with the living-body-attachable device 5. In the present embodiment, the communication section 40 receives a first feature amount that is calculated by each potential measurement section 3 and transmitted from the living-body-attachable device 5.

The second signal processor 41B calculates second feature amounts used to estimate a state of the human 2, using first feature amounts respectively calculated by the first signal processors 35 respectively included in the plurality of potential measurement sections 3.

Specifically, the second signal processor 41B compares magnitudes of first feature amounts respectively calculated by the plurality of potential measurement sections 3, and sets the first feature amount exhibiting a smallest value to be a reference feature amount.

Next, the second signal processor 41B calculates a second feature amount by subtracting the reference feature amount from each of the first feature amounts respectively calculated by the potential measurement sections 3 other than the potential measurement section 3 calculating the first feature amount set to be the reference feature amount.

For example, it is assumed that two potential measurement sections are a first potential measurement section and a second potential measurement section, and that a first feature amount calculated by the second potential measurement section is set to be a reference feature amount. The second signal processor 41B calculates a second feature amount by subtracting the reference feature amount from the first feature amount calculated by the first potential measurement section. Note that an example in which the number of potential measurement sections is two, has been described here. However, at least three potential measurement sections may be provided. In this case, the second feature amount can be calculated in a similar manner.

The state estimator 42 estimates a state of the human 2 on the basis of the second feature amount. State information regarding the estimated state of the human 2 is transmitted to the provision section 43 and the storage 44.

### [Flow of Processing Performed by Biological Information Acquiring System]

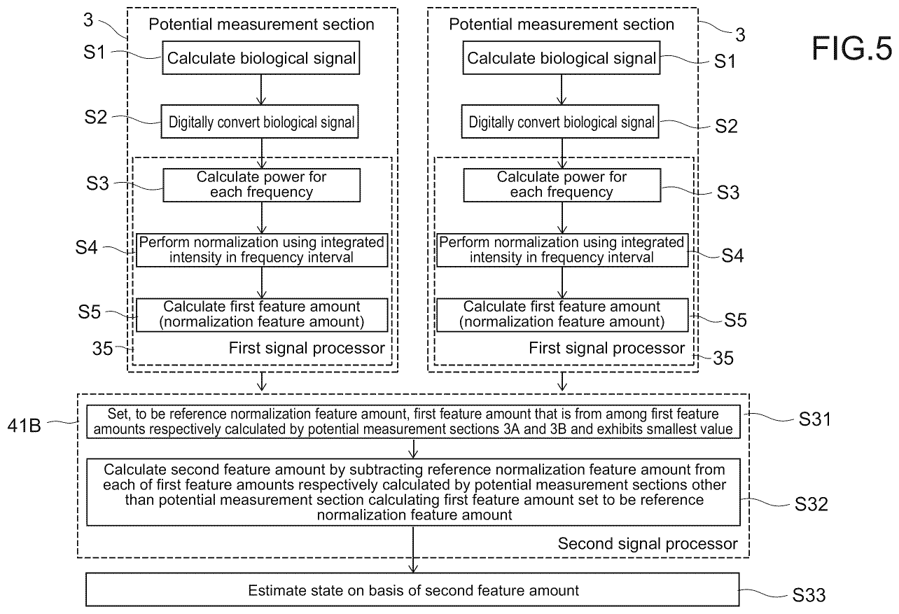

A flow of processing performed by the biological information acquiring system 1B described above is described with reference to FIG. 5.

In each potential measurement section 3 of the living-body-attachable device 5, the potential of the measurement electrode 30 and the potential of the reference electrode 31 are measured, and the analog front-end section 33 generates (S1) a brain-wave signal from a difference between the potential of the measurement electrode 30 and the potential of the reference electrode 31, as illustrated in FIG. 5.

Next, the analog-digital converter 34 digitally converts (S2) the brain-wave signal.

Next, the first signal processor 35 calculates (S3) power for each frequency from the digitally converted brain-wave signal.

Next, the first signal processor 35 normalizes (S4) the calculated power for each frequency (a power distribution) using an integrated intensity in a specified frequency interval to calculate (S5) a first feature amount for a normalized intensity.

The first feature amount calculated by each potential measurement section 3 is transmitted to the external apparatus 6B through the communication section 36.

As described above, information output from the living-body-attachable device 5 to the external apparatus 6B is digitized information.

The external apparatus 6B receives, through the communication section 40, the first feature amount calculated by each potential measurement section 3.

The second signal processor 41B of the external apparatus 6B compares magnitudes of the first feature amounts respectively calculated by the plurality of potential measurement sections 3, and sets the first feature amount exhibiting a smallest value to be a reference feature amount (S31).

Next, the second signal processor 41B calculates a second feature amount by subtracting the reference feature amount from each of the first feature amounts respectively calculated by the potential measurement sections 3 other than the potential measurement section 3 calculating the first feature amount set to be the reference feature amount (S32).

Next, the state estimator 42 of the external apparatus 6B estimates a state of the human 2 on the basis of the second feature amount (S33).

As described above, in the biological information acquiring system 1B according to the present embodiment, a brain-wave signal (a biological signal) that is acquired by each of the plurality of potential measurement sections 3 of the living-body-attachable device 5 is digitized to be converted into power for each frequency. Then, the power is normalized to calculate a first feature amount (a normalization feature amount), and the calculated first feature amount is transmitted to the external apparatus 6B.

Then, the second signal processor 41B of the external apparatus 6B calculates a second feature amount used to estimate a state of the human 2 using pieces of digitized biological information (the first feature amounts) respectively acquired by a plurality of potential measurement sections 3 of which reference electrodes are situated at different positions.

As in the case of the first embodiment, such a configuration makes it possible to increase an S/N ratio by performing normalization processing even if a level (an intensity) of a brain-wave signal acquired by each potential measurement section 3 is low. Further, a second feature amount is calculated by combining pieces of information respectively acquired by a plurality of potential measurement sections 3 of which reference electrodes are situated at different positions. This makes it possible to estimate a state of the human 2 with a high degree of accuracy.

Further, each potential measurement section 3 of the living-body-attachable device 5 digitally communicates with the external apparatus 6B. Consequently, the potential measurement sections 3 each being an individual unit can be made separate from each other, and the usability can be improved.

Further, a degree of freedom in design of the living-body-attachable device 5 can be increased. This makes it possible to make the living-body-attachable device 5 smaller in size, and thus to improve the usability. Further, the increase in the degree of freedom in design makes it possible to design a living-body-attachable device suitable for measurement as appropriate by, for example, making the device smaller in size, such that burdens imposed on a measurement target when the living-body-attachable device is attached to the measurement target, are reduced.

Furthermore, when a feature amount exhibiting a large change is compared with a first feature amount exhibiting a smallest change using the first feature amount exhibiting a smallest change as a reference, as in the case of the present embodiment, this makes it possible to estimate a state of a living body with a higher degree of accuracy.

### Fourth Embodiment

### [Configuration of Biological Information Acquiring System]

The example in which the first signal processor performs normalization processing has been described in the first embodiment. When, for example, the living-body-attachable device has a configuration in which a distance between the measurement electrode and the reference electrode is sufficiently large so that a level of an acquired brain-wave signal can be sufficiently high, the second signal processor may perform normalization processing, as in the case of the present embodiment.

In a biological information acquiring system according to the present embodiment, the first signal processor included in each of the plurality of potential measurement sections calculates a first feature amount using a digitally converted brain-wave signal, and the second signal processor calculates a second feature amount used to estimate a state of a human using the first feature amounts calculated by the respective potential measurement sections, as in the case of the first embodiment.

FIGS. 1 and 2 each schematically illustrate a configuration of a biological information acquiring system 1C according to the present embodiment.

As illustrated in FIG. 1, the biological information acquiring system 1C according to the present embodiment includes a living-body-attachable device 5C that serves as a biological information acquiring apparatus, and an external apparatus 6C that serves as an information processing apparatus.

The living-body-attachable device 5C is attachable to the head of the target-measurement human 2, more particularly the ear of the human 2. The living-body-attachable device 5C includes a plurality of potential measurement sections 3C. Each potential measurement section 3 includes a first signal processor 35C described later.

The external apparatus 6C is an apparatus that is different from the living-body-attachable device 5C. Examples of the external apparatus 6C include other terminals such as a smartphone and a personal computer (PC), and a server apparatus and a cloud that are in a network. The external apparatus 6C includes a second signal processor 41C described later.

### [Configuration of Living-Body-Attachable Device (Biological Information Acquiring Apparatus)]

As illustrated in FIGS. 1 and 2, the living-body-attachable device 5C includes the plurality of potential measurement sections 3C. The potential measurement sections 3C have the same configuration.

Each of the plurality of potential measurement sections 3C is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35C, the communication section 36, and the power-supply section 37.

As illustrated in FIG. 2, the measurement electrode 30, the reference electrode 31, and the bias electrode 32 are arranged, for example, in or near the ear of the human 2. In the example illustrated in FIG. 2, the potential measurement section 3C of the living-body-attachable device 5C is arranged on each of right and left ears of the human 2, and brain-wave information regarding brain waves between the right and left ears can be acquired.

The first signal processor 35C calculates, as a first feature amount, power for each frequency from a brain-wave signal that is generated by the analog front-end section 33 and digitally converted by the analog-digital converter 34.

The communication section 36 communicates with the external apparatus 6. In the present embodiment, the communication section 36 transmits, to the external apparatus 6C, a first feature amount (information regarding the power for each frequency) calculated by the first signal processor 35C.

### [Configuration of External Apparatus (Information Processing Apparatus)]

As illustrated in FIG. 1, the external apparatus 6C includes the communication section 40, the second signal processor 41C, the state estimator 42, the provision section 43, and the storage 44.

The communication section 40 communicates with the living-body-attachable device 5C. In the present embodiment, the communication section 40 receives information regarding power calculated for each frequency from a digitally converted brain-wave signal (a first feature amount), the power being calculated by each potential measurement section 3C and transmitted from the living-body-attachable device 5C.

The second signal processor 41C calculates a second feature amount used to estimate a state of the human 2, using first feature amounts respectively calculated by the first signal processors 35C respectively included in the plurality of potential measurement sections 3C.

Specifically, the second signal processor 41C calculates an intermediate feature amount by performing signal averaging on the first feature amounts respectively calculated by the plurality of potential measurement sections 3C.

Next, the second signal processor 41C normalizes the intermediate feature amount using an integrated intensity in a specified frequency interval to calculate a second feature amount for a normalized intensity.

The state estimator 42 estimates a state of the human 2 on the basis of the second feature amount. State information regarding the estimated state of the human 2 is transmitted to the provision section 43 and the storage 44.

### [Flow of Processing Performed by Biological Information Acquiring System]

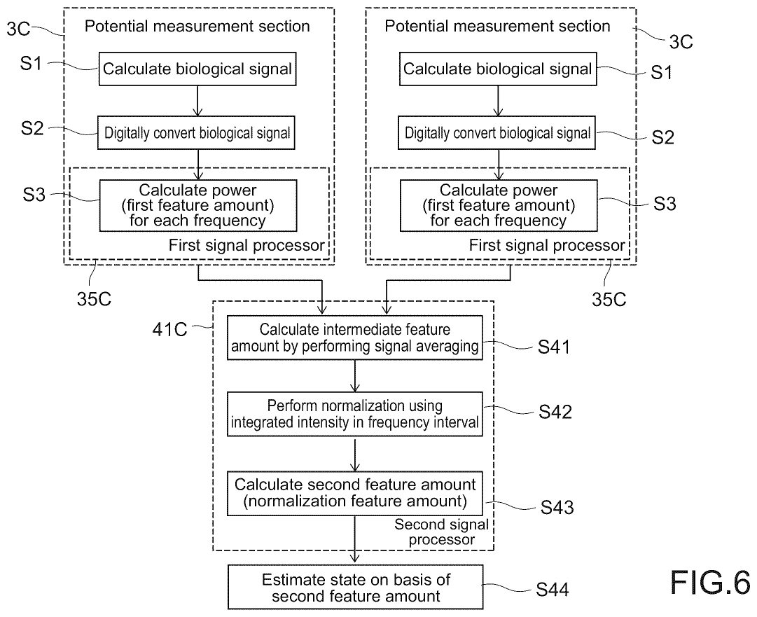

A flow of processing performed by the biological information acquiring system 1C described above is described with reference to FIG. 6.

In each potential measurement section 3C of the living-body-attachable device 5C, the potential of the measurement electrode 30 and the potential of the reference electrode 31 are measured, and the analog front-end section 33 generates (S1) a brain-wave signal from a difference between the potential of the measurement electrode 30 and the potential of the reference electrode 31, as illustrated in FIG. 6.

Next, the analog-digital converter 34 digitally converts (S2) the brain-wave signal.

Next, the first signal processor 35C calculates (S3) power for each frequency from the digitally converted brain-wave signal.

The first feature amount calculated by each potential measurement section 3C is transmitted to the external apparatus 6C through the communication section 36.

As described above, information output from the living-body-attachable device 5C to the external apparatus 6C is digitized information.

The external apparatus 6C receives, through the communication section 40, the first feature amount calculated by each potential measurement section 3C.

The second signal processor 41C of the external apparatus 6C calculates (S41) an intermediate feature amount by performing signal averaging on the first feature amounts respectively acquired by the plurality of potential measurement sections 3C.

Next, the second signal processor 41C normalizes (S42) the intermediate feature amount using an integrated intensity in a specified frequency interval to calculate (S43) a first feature amount for a normalized intensity.

Next, the state estimator 42 of the external apparatus 6C estimates a state of the human 2 on the basis of the second feature amount (S44).

As described above, in the biological information acquiring system 1C according to the present embodiment, a brain-wave signal (a biological signal) that is acquired by each of the plurality of potential measurement sections 3C of the living-body-attachable device 5C is digitized to be converted into power for each frequency (a first feature amount). Then, the power is transmitted to the external apparatus 6C.

Then, the second signal processor 41C of the external apparatus 6C calculates a second feature amount used to estimate a state of the human (a living body) 2 using pieces of digitized biological information (pieces of information regarding the power for each frequency that is obtained by the conversion (the first feature amounts)) respectively acquired by a plurality of potential measurement sections 3C of which reference electrodes are situated at different positions.

According to such a configuration, a second feature amount is calculated by combining pieces of information respectively acquired by a plurality of potential measurement sections 3 of which reference electrodes are situated at different positions. This makes it possible to estimate a state of the human 2 with a high degree of accuracy.

### Other Embodiments

Fifth to ninth embodiments and the tenth embodiment are described below.

The examples of mounting patterns in which the first signal processor is provided to the living-body-attachable device and the second signal processor is provided to the external apparatus, have been described in the embodiments above. However, the present technology is not limited thereto. Examples of other mounting patterns are described in the fifth to ninth embodiments.

With respect to the signal processes performed by the first and second signal processors, the first to fourth embodiments described above can be applied to the fifth to ninth embodiments. The fifth to ninth embodiments are described focused on a point different from that of the first embodiment. A structural element that is similar to the structural element described in the first embodiment is denoted by a reference numeral similar to the reference numeral used in the first embodiment, and a description thereof may be omitted.

Which mounting pattern is to be selected can be determined from a synthetic perspective in consideration of, for example, power consumption, the size of a device, and an environment in which the device is used. For example, a mounting pattern in which the living-body-attachable device only includes the potential measurement sections and the second signal processor is provided to the external apparatus, is favorably adopted from a usability perspective, as in the case the first to fourth embodiments and the eighth and ninth embodiments described later.

In a biological information acquiring system according to each of the fifth to ninth embodiments, the first signal processor included in each of the plurality of potential measurement sections calculates a first feature amount using a digitally converted brain-wave signal, and the second signal processor calculates a second feature amount used to estimate a state of a human, using the first feature amounts calculated by the respective potential measurement sections, as in the case of the first embodiment.

Further, an example of a system configuration in which a first feature amount is calculated using PCA or ICA is described in the tenth embodiment. The tenth embodiment is described focused on a point different from that of the first embodiment. A structural element that is similar to the structural element described in the first embodiment is denoted by a reference numeral similar to the reference numeral used in the first embodiment, and a description thereof may be omitted. Further, an example of a mounting pattern similar to a mounting pattern according to the first embodiment, that is, an example of a mounting pattern in which the first signal processor is provided to the living-body-attachable device and the second signal processor is provided to the external apparatus, is described in the tenth embodiment. However, mounting patterns that are described in the fifth to ninth embodiments can also be applied.

Note that the configuration in which each potential measurement section includes a plurality of measurement electrodes can be applied to not only the case in which PCA or ICA is used as the method for calculating a first feature amount, but also the case in which another calculation method such as FFT is adopted.

The respective embodiments are described below.

### Fifth Embodiment

### [Configuration of Biological Information Acquiring System]

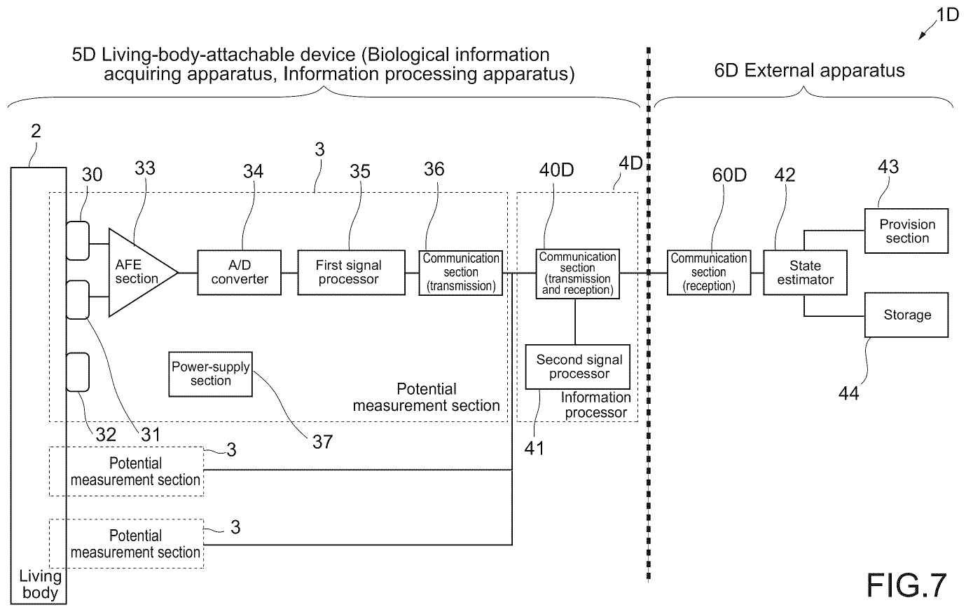

FIG. 7 schematically illustrates a configuration of a biological information acquiring system 1D according to the present embodiment.

As illustrated in FIG. 7, an example in which the second signal processor 41 is provided to a living-body-attachable device 5D is described in the present embodiment. In the biological information acquiring system 1D, the living-body-attachable device 5D serves as both a biological information acquiring apparatus and an information processing apparatus.

As used herein, the “biological information acquiring apparatus” refers to an apparatus that at least serves to acquire a biological signal (a brain-wave signal in the present embodiment), to digitally convert the biological signal, and to calculate a first feature amount using the digitally convert biological signal. The calculation of the first feature amount is performed by the first signal processor 35.

As used herein, the “information processing apparatus” refers to an apparatus that serves to calculate a second feature amount used to estimate a state of a living body, using a first feature amount calculated from a digitally convert biological signal. The calculation of the second feature amount is performed by the second signal processor 41.

As illustrated in FIG. 7, the biological information acquiring system 1D includes the living-body-attachable device 5D serving as a biological information acquiring apparatus and an information processing apparatus, and an external apparatus 6D.

The living-body-attachable device 5D is attachable to the head of the target-measurement human 2, more particularly the ear of the human 2. The living-body-attachable device 5D includes a plurality of potential measurement sections 3 and an information processor 4D. Each potential measurement section 3 includes the first signal processor 35. The information processor 4D includes the second signal processor 41.

The external apparatus 6D is an apparatus that is different from the living-body-attachable device 5D. Examples of the external apparatus 6D include other terminals such as a smartphone and a personal computer (PC), and a server apparatus and a cloud that are in a network.

### [Configuration of Living-Body-Attachable Device (Biological Information Acquiring Apparatus, Information Processing Apparatus)]

As illustrated in FIG. 7, the living-body-attachable device 5D includes the plurality of potential measurement sections 3 and the information processor 4D.

The potential measurement sections 3 have the same configuration. FIG. 7 illustrates the living-body-attachable device 5D including three potential measurement sections 3. However, it is sufficient if at least two potential measurement sections 3 are provided.

### Configuration of Potential Measurement Section

As illustrated in FIG. 7, each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates a first feature amount using a digitally converted brain-wave signal.

The communication section 36 communicates with the information processor 4D. In the present embodiment, the communication section 36 transmits, to the information processor 4D, a first feature amount calculated by the first signal processor 35.

### Configuration of Information Processor

As illustrated in FIG. 7, the information processor 4D is a unit that includes a communication section 40D and the second signal processor 41.

The communication section 40D communicates with the respective potential measurement sections 3 and the external apparatus 6D. In the present embodiment, the communication section 40D receives a first feature amount calculated by each potential measurement section 3. Further, the communication section 40D transmits, to the external apparatus 6D, a second feature amount calculated by the second signal processor 41. The communication section 36, the communication section 40D, and a communication section 60D described later may perform communication by wire or wirelessly, but favorably perform wireless communication from a usability perspective.

The second signal processor 41 calculates a second feature amount using the first feature amounts respectively calculated by the plurality of potential measurement sections 3.

Note that the example in which, in the living-body-attachable device 5D, the information processor 4D is separate from the respective potential measurement sections 3 each being a unit, has been described in the present embodiment. However, one of the plurality of potential measurement sections may include the information processor 4D.

### [Configuration of External Apparatus]

As illustrated in FIG. 7, the external apparatus 6D includes the communication section 60D, the state estimator 42, the provision section 43, and the storage 44.

The communication section 60D communicates with the living-body-attachable device 5D. In the present embodiment, the communication section 60D receives a second feature amount from the living-body-attachable device 5D.

The state estimator 42 estimates a state of the human 2 on the basis of the second feature amount. State information regarding the estimated state of the human 2 is transmitted to the provision section 43 and the storage 44.

As described above, the second signal processor calculating a second feature amount used to estimate a state of a living body may be provided to the living-body-attachable device.

### Sixth Embodiment

### [Configuration of Biological Information Acquiring System]

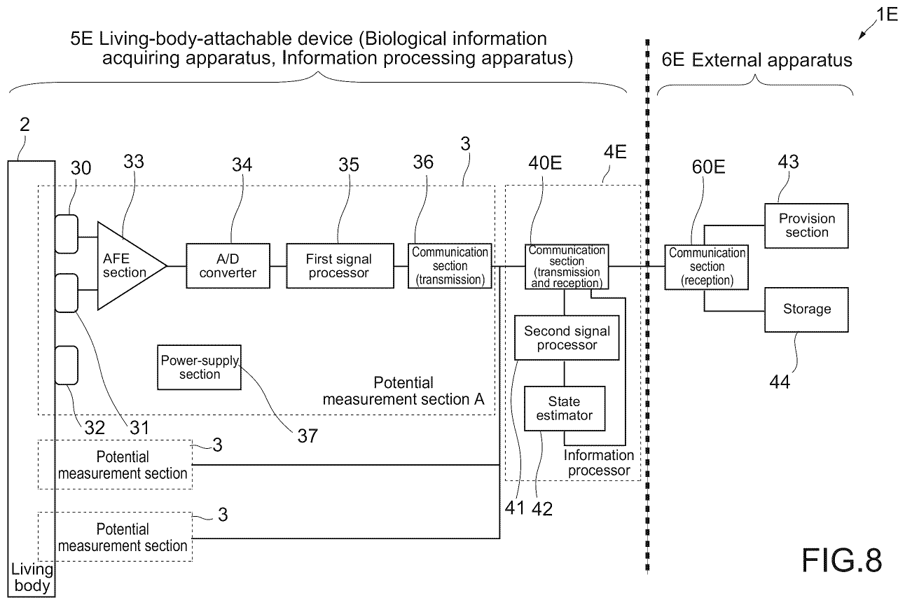

FIG. 8 schematically illustrates a configuration of a biological information acquiring system 1E according to the present embodiment.

As illustrated in FIG. 8, an example in which the second signal processor 41 and the state estimator 42 are provided to a living-body-attachable device is described in the present embodiment. In the biological information acquiring system 1E according to the present embodiment, a living-body-attachable device 5E serves as both a biological information acquiring apparatus and an information processing apparatus.

As illustrated in FIG. 8, the biological information acquiring system 1E includes the living-body-attachable device 5E serving as a biological information acquiring apparatus and an information processing apparatus, and an external apparatus 6E.

The living-body-attachable device 5E is attachable to the head of the target-measurement human 2, more particularly the ear of the human 2. The living-body-attachable device 5E includes a plurality of potential measurement sections 3 and an information processor 4E. Each potential measurement section 3 includes the first signal processor 35. The information processor 4E includes the second signal processor 41 and the state estimator 42.

The external apparatus 6E is an apparatus that is different from the living-body-attachable device 5E. Examples of the external apparatus 6E include other terminals such as a smartphone and a personal computer (PC), and a server apparatus and a cloud that are in a network.

### [Configuration of Living-Body-Attachable Device (Biological Information Acquiring Apparatus)]

As illustrated in FIG. 8, the living-body-attachable device 5E includes the plurality of potential measurement sections 3 and the information processor 4E.

The potential measurement sections 3 have the same configuration. FIG. 8 illustrates the living-body-attachable device 5E including three potential measurement sections 3. However, it is sufficient if at least two potential measurement sections 3 are provided.

### Configuration of Potential Measurement Section

As illustrated in FIG. 8, each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates a first feature amount using a digitally converted brain-wave signal.

The communication section 36 communicates with the information processor 4E. In the present embodiment, the communication section 36 transmits, to the information processor 4E, a first feature amount calculated by the first signal processor 35.

### Configuration of Information Processor

As illustrated in FIG. 8, the information processor 4E is a unit that includes a communication section 40E, the second signal processor 41, and the state estimator 42.

The communication section 40E communicates with the respective potential measurement sections 3 and the external apparatus 6E. In the present embodiment, the communication section 40E receives a first feature amount calculated by each potential measurement section 3. Further, the communication section 40D transmits state information regarding a state of the human 2 to the external apparatus 6E, the state of the human 2 being estimated by the state estimator 42 on the basis of a second feature amount calculated by the second signal processor 41. The communication section 36, the communication section 40E, and a communication section 60E described later may perform communication by wire or wirelessly, but favorably perform wireless communication from a usability perspective.

The second signal processor 41 calculates a second feature amount using the first feature amounts respectively calculated by the plurality of potential measurement sections 3.

The state estimator 42 estimates a state of the human 2 on the basis of the second feature amount. State information regarding the estimated state of the human 2 is transmitted to the external apparatus 6E through the communication section 40E.

Note that the example in which, in the living-body-attachable device 5E, the information processor 4E is separate from the respective potential measurement sections 3 each being a unit, has been described in the present embodiment. However, one of the plurality of potential measurement sections may include the information processor 4E.

### [Configuration of External Apparatus (Information Processing Apparatus)]

As illustrated in FIG. 8, the external apparatus 6E includes the communication section 60E, the provision section 43, and the storage 44.

The communication section 60E communicates with the living-body-attachable device 5E. In the present embodiment, the communication section 60E receives state information regarding a state of the human 2, the state information being transmitted from the living-body-attachable device 5E.

The received state information regarding a state of the human 2 is transmitted to the provision section 43 and the storage 44.

As described above, the second signal processor calculating a second feature amount used to estimate a state of a living body, and the state estimator 42 may be provided to the living-body-attachable device.

### Seventh Embodiment

### [Configuration of Biological Information Acquiring System]

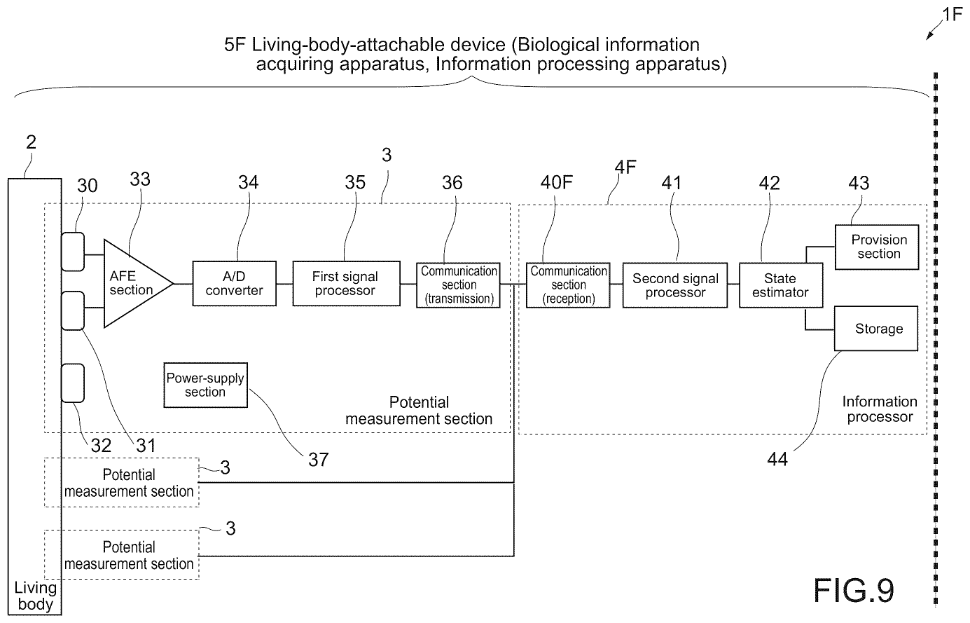

FIG. 9 schematically illustrates a configuration of a biological information acquiring system 1F according to the present embodiment.

As illustrated in FIG. 9, an example in which the second signal processor 41, the state estimator 42, the provision section 43, and the storage 44 are provided to a living-body-attachable device 5F is described in the present embodiment. In the biological information acquiring system 1F according to the present embodiment, an external apparatus is not provided, and the living-body-attachable device 5F serves as both a biological information acquiring apparatus and an information processing apparatus.

As illustrated in FIG. 9, the biological information acquiring system 1F includes the living-body-attachable device 5F serving as a biological information acquiring apparatus and an information processing apparatus. The living-body-attachable device 5F is attachable to the head of the target-measurement human 2, more particularly the ear of the human 2.

### [Configuration of Living-Body-Attachable Device (Biological Information Acquiring Apparatus, Information Processing Apparatus)]

As illustrated in FIG. 9, the living-body-attachable device 5F includes the plurality of potential measurement sections 3 and the information processor 4F.

### Configuration of Potential Measurement Section

As illustrated in FIG. 9, each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates a first feature amount using a digitally converted brain-wave signal.

The communication section 36 communicates with the information processor 4F. In the present embodiment, the communication section 36 transmits, to the information processor 4F, a first feature amount calculated by the first signal processor 35.

### Configuration of Information Processor

As illustrated in FIG. 9, the information processor 4F is a unit that includes a communication section 40F, the second signal processor 41, the state estimator 42, the provision section 43, and the storage 44.

The communication section 40F communicates with the respective potential measurement sections 3. In the present embodiment, the communication section 40F receives a first feature amount calculated by each potential measurement section 3. The communication section 36 and the communication section 40F may perform communication by wire or wirelessly, but favorably perform wireless communication from a usability perspective.

The second signal processor 41 calculates a second feature amount using the first feature amounts respectively calculated by the plurality of potential measurement sections 3.

The state estimator 42 estimates a state of the human 2 on the basis of the second feature amount. State information regarding the estimated state of the human 2 is transmitted to the provision section 43 and the storage 44.

Note that the example in which, in the living-body-attachable device 5F, the information processor 4F is separate from the respective potential measurement sections 3 each being a unit, has been described in the present embodiment. However, one of the plurality of potential measurement sections may include the information processor 4F.

As described above, the second signal processor calculating a second feature amount used to estimate a state of a living body, the state estimator 42, the provision section 43, and the storage 44 may be provided to the living-body-attachable device.

### Eighth Embodiment

### [Configuration of Biological Information Acquiring System]

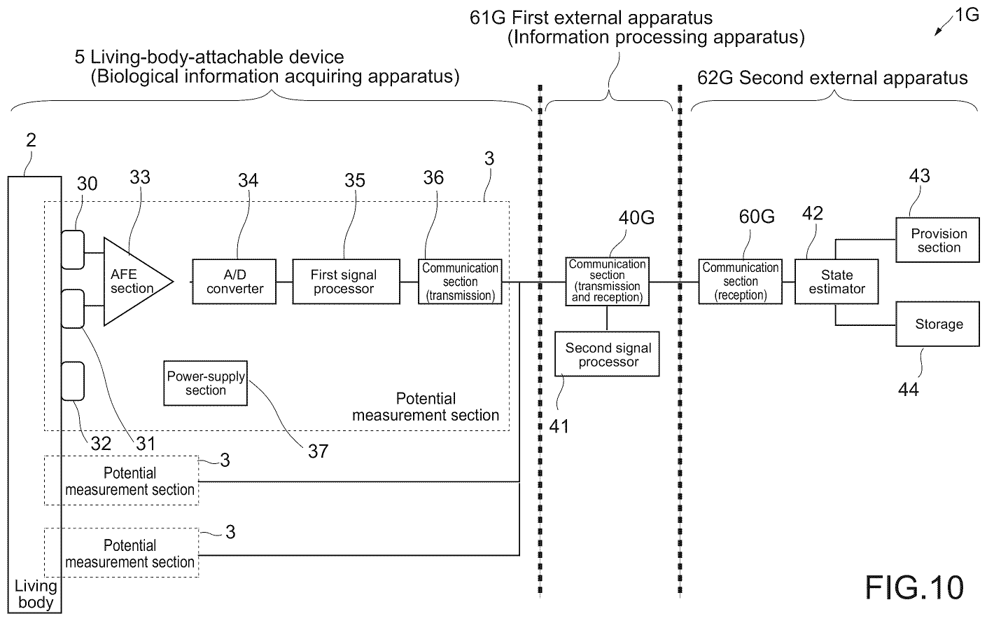

FIG. 10 schematically illustrates a configuration of a biological information acquiring system 1G according to the present embodiment.

As illustrated in FIG. 10, an example in which the second signal processor 41 is provided to a first external apparatus 61G, and the state estimator 42, the provision section 43, and storage 44 are provided to a second external apparatus 62G, is described in the present embodiment.

In the biological information acquiring system 1G, the living-body-attachable device 5 serves as a biological information acquiring apparatus, and the first external apparatus 61G serves as an information processing apparatus.

As illustrated in FIG. 10, the biological information acquiring system 1G includes the living-body-attachable device 5 serving as a biological information acquiring apparatus, the first external apparatus 61G serving as an information processing apparatus, and the second external apparatus 62G.

The living-body-attachable device 5 is attachable to the head of the target-measurement human 2, more particularly the ear of the human 2. The living-body-attachable device 5 includes a plurality of potential measurement sections 3. Each potential measurement section 3 includes the first signal processor 35.

The first external apparatus 61G and the second external apparatus 62G are apparatuses that are different from the living-body-attachable device 5. Examples of the first external apparatus 61G and the second external apparatus 62G include other terminals such as a smartphone and a personal computer (PC), and a server apparatus and a cloud that are in a network.

The first external apparatus 61G includes the second signal processor 41. The second external apparatus 62G includes the state estimator 42, the provision section 43, and the storage 44.

### [Configuration of Living-Body-Attachable Device (Biological Information Acquiring Apparatus)]

As illustrated in FIG. 10, the living-body-attachable device 5 includes the plurality of potential measurement sections 3.

Each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates a first feature amount using a digitally converted brain-wave signal.

The communication section 36 communicates with the first external apparatus 61G. In the present embodiment, the communication section 36 transmits, to the first external apparatus 61G, a first feature amount calculated by the first signal processor 35.

### [Configuration of First External Apparatus (Information Processing Apparatus)]

As illustrated in FIG. 10, the first external apparatus 61G includes a communication section 40G and the second signal processor 41.

The communication section 40G communicates with the respective potential measurement sections 3 and the second external apparatus 62G. In the present embodiment, the communication section 40G receives a first feature amount calculated by each potential measurement section 3. Further, the communication section 40G transmits, to the second external apparatus 62G, a second feature amount calculated by the second signal processor 41. The communication section 36, the communication section 40G, and a communication section 60G described later may perform communication by wire or wirelessly, but favorably perform wireless communication from a usability perspective.

The second signal processor 41 calculates a second feature amount using the first feature amounts respectively calculated by the plurality of potential measurement sections 3.

### [Configuration of Second External Apparatus]

As illustrated in FIG. 10, the second external apparatus 62G includes the communication section 60G, the state estimator 42, the provision section 43, and the storage 44.

The communication section 60G communicates with the first external apparatus 61G. In the present embodiment, the communication section 60G receives a second feature amount from the first external apparatus 61G.

The state estimator 42 estimates a state of the human 2 on the basis of the second feature amount. State information regarding the estimated state of the human 2 is transmitted to the provision section 43 and the storage 44.

As described above, a plurality of apparatuses (external apparatuses) each being different from the living-body-attachable device may be provided to the biological information acquiring system.

### Ninth Embodiment

### [Configuration of Biological Information Acquiring System]

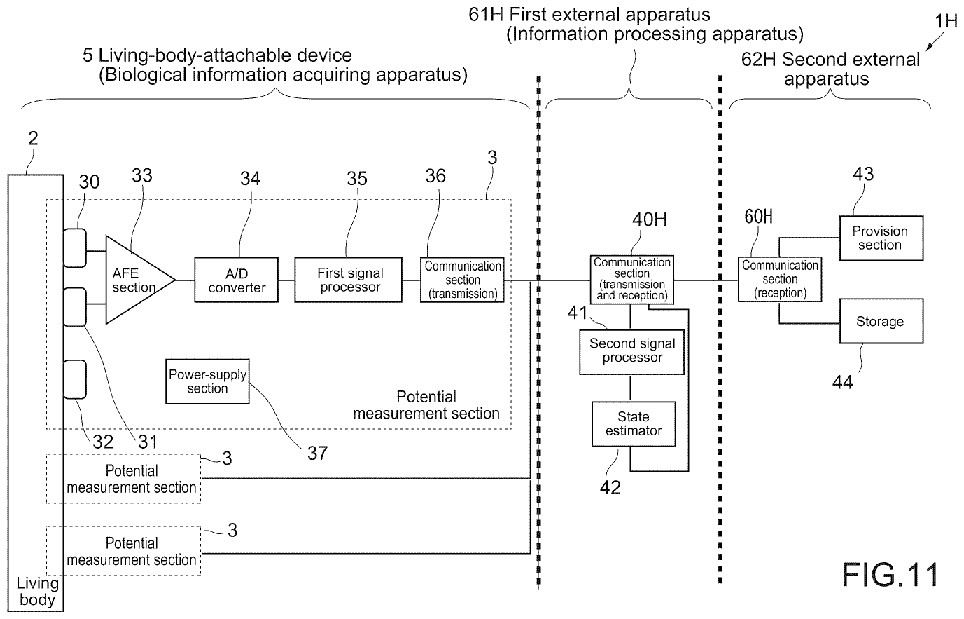

FIG. 11 schematically illustrates a configuration of a biological information acquiring system 1H according to the present embodiment.

As illustrated in FIG. 11, an example in which the second signal processor 41 and the state estimator 42 are provided to a first external apparatus 61H, and the provision section 43 and storage 44 are provided to a second external apparatus 62H, is described in the present embodiment.

In the biological information acquiring system 1H, the living-body-attachable device 5 serves as a biological information acquiring apparatus, and the first external apparatus 61H serves as an information processing apparatus.

As illustrated in FIG. 11, the biological information acquiring system 1H includes the living-body-attachable device 5 serving as a biological information acquiring apparatus, the first external apparatus 61H serving as an information processing apparatus, and the second external apparatus 62H.

The living-body-attachable device 5 is attachable to the head of the target-measurement human 2, more particularly the ear of the human 2. The living-body-attachable device 5 includes a plurality of potential measurement sections 3. Each potential measurement section 3 includes the first signal processor 35.

The first external apparatus 61H and the second external apparatus 62H are apparatuses that are different from the living-body-attachable device 5. Examples of the first external apparatus 61H and the second external apparatus 62H include other terminals such as a smartphone and a personal computer (PC), and a server apparatus and a cloud that are in a network.

The first external apparatus 61H includes the second signal processor 41 and the state estimator 42. The second external apparatus 62H includes the provision section 43 and the storage 44.

### [Configuration of Living-Body-Attachable Device (Biological Information Acquiring Apparatus)]

As illustrated in FIG. 11, the living-body-attachable device 5 includes the plurality of potential measurement sections 3.

Each of the plurality of potential measurement sections 3 is a unit that includes the measurement electrode 30, the reference electrode 31, the bias electrode 32, the analog front-end section (the AFE section) 33 serving as a biological signal generator, the analog-digital converter (the A/D converter) 34, the first signal processor 35, the communication section 36, and the power-supply section 37.

The first signal processor 35 calculates a first feature amount using a digitally converted brain-wave signal.

The communication section 36 communicates with the first external apparatus 61H. In the present embodiment, the communication section 36 transmits, to the first external apparatus 61H, a first feature amount calculated by the first signal processor 35.

### [Configuration of First External Apparatus (Information Processing Apparatus)]

As illustrated in FIG. 11, the first external apparatus 61H includes a communication section 40H, the second signal processor 41, and the state estimator 42.

The communication section 40H communicates with the respective potential measurement sections 3 and the second external apparatus 62H. In the present embodiment, the communication section 40H receives a first feature amount calculated by each potential measurement section 3. Further, the communication section 40H transmits state information regarding a state of the human 2 to the second external apparatus 6H, the state of the human 2 being estimated by the state estimator 42 on the basis of a second feature amount calculated by the second signal processor 41. The communication section 36, the communication section 40H, and a communication section 60H described later may perform communication by wire or wirelessly, but favorably perform wireless communication from a usability perspective.

The second signal processor 41 calculates a second feature amount using the first feature amounts respectively calculated by the plurality of potential measurement sections 3.

The state estimator 42 estimates a state of the human 2 on the basis of the second feature amount.

### [Configuration of Second External Apparatus]

As illustrated in FIG. 11, the second external apparatus 62H includes the communication section 60H, the provision section 43, and the storage 44.

The communication section 60H communicates with the first external apparatus 61H. In the present embodiment, the communication section 60H receives state information regarding a state of the human 2, the state information being transmitted by the first external apparatus 61H. The state information regarding the state of the human 2 is transmitted to the provision section 43 and the storage 44.

As described above, a plurality of apparatuses (external apparatuses) each being different from the living-body-attachable device may be provided to the biological information acquiring system.

### Tenth Embodiment

FIG. 12 schematically illustrates a configuration of a biological information acquiring system 1J according to the tenth embodiment.

As illustrated in FIG. 12, the biological information acquiring system 1J includes a living-body-attachable device 5J that serves as a biological information acquiring apparatus, and an external apparatus 6J that serves as an information processing apparatus.

The living-body-attachable device 5J is attachable to the head of the target-measurement human (a living body) 2, more particularly the ear of the human 2. The living-body-attachable device 5J includes a plurality of potential measurement sections 3J. Each potential measurement section 3J includes a first signal processor 35J described later.

The external apparatus 6J is an apparatus that is different from the living-body-attachable device 5J. Examples of the external apparatus 6J include other terminals such as a smartphone and a personal computer (PC), and a server apparatus and a cloud that are in a network. The external apparatus 6J includes a second signal processor 41J described later.

In the biological information acquiring system 1J, a second feature amount used to estimate a state of the human 2 is calculated using brain-wave signals (biological signals) respectively acquired by the plurality of potential measurement sections 3J, as in the cases of the other embodiments described above.

### [Configuration of Living-Body-Attachable Device (Biological Information Acquiring Apparatus)]

As illustrated in FIG. 12, the living-body-attachable device 5J includes the plurality of potential measurement sections 3J. The potential measurement sections 3J have the same configuration.

For convenience, FIG. 12 illustrates the living-body-attachable device 5J including three potential measurement sections 3J. However, the number of potential measurement sections 3 is not limited thereto, and it is sufficient if at least two potential measurement sections 3J are provided.

Each of the plurality of potential measurement sections 3 is a unit that includes a plurality of measurement electrodes 30, the reference electrode 31, the bias electrode 32, the analog front-end sections 33 each provided for a corresponding one of the plurality of measurement electrodes 30, the analog-digital converters 34 each provided for a corresponding one of the plurality of measurement electrodes 30, the first signal processor 35J, the communication section 36, and the power-supply section 37.

When a first feature amount is calculated using PCA or ICA, each potential measurement section 3J is configured to include a plurality of measurement electrodes 30, as illustrated in FIG. 12. The configuration in which each potential measurement section 3J includes a plurality of measurement electrodes 30 enables each potential measurement section 3J to acquire a plurality of brain-wave signals (biological signals) that corresponds to a plurality of measurement signals. Note that each potential measurement section 3J may also be configured to include a plurality of measurement electrodes 30 when a first feature amount is calculated using FFT.

For convenience, FIG. 12 illustrates an example of providing three measurement electrodes 30. However, the number of measurement electrodes 30 is not limited thereto, and it is sufficient if at least two measurement electrodes 30 are provided.

The measurement electrode 30, the reference electrode 31, and the bias electrode 32 are arranged, for example, in or near the ear of the human 2.

The first signal processor 35J calculates a first feature amount using a digitally converted brain-wave signal. In the present embodiment, a first feature amount is calculated using PCA or ICA.

In each potential measurement section 3J, each analog front-end section 33 generates a brain-wave signal (a biological signal) from a difference between a potential of the measurement electrode 30 corresponding to the analog front-end section 33, and a potential of the reference electrode 31, as illustrated in FIG. 12. The brain-wave signal generated by each analog front-end section 33 is digitally converted by the analog-digital converter 34 corresponding to the analog front-end section 33, and the digitally converted brain-wave signal is output to the first signal processor 35J.

The first signal processor 35J calculates a first feature amount using a plurality of the digitally converted brain-wave signals by use of PCA or ICA. The calculated first feature amount is transmitted to the external apparatus 6J by the communication section 36.

### [Configuration of External Apparatus (Information Processing Apparatus)]

As illustrated in FIG. 12, the external apparatus 6J includes the communication section 40, the second signal processor 41J, the state estimator 42, the provision section 43, and the storage 44.

The communication section 40 communicates with the living-body-attachable device 5J. In the present embodiment, the communication section 40 receives a first feature amount that is calculated by each potential measurement section 3 and transmitted from the living-body-attachable device 5J.

The second signal processor 41 calculates a second feature amount, using first feature amounts that are respectively calculated by the plurality of potential measurement sections 3 and received from the living-body-attachable device 5J through the communication section 40.

For example, specifically, the second signal processor 41J may compare magnitudes of first feature amounts respectively calculated by the plurality of potential measurement sections 3J, may set the first feature amount exhibiting a smallest value to be a reference feature amount, and may calculate a second feature amount by performing signal averaging on the reference feature amount and each of the first feature amounts respectively calculated by the potential measurement sections 3J other than the potential measurement section 3J calculating the first feature amount set to be the reference feature amount.

Further, the second signal processor 41J may compare magnitudes of first feature amounts respectively calculated by the plurality of potential measurement sections 3J, may set the first feature amount exhibiting a smallest value to be a reference feature amount, and may calculate a second feature amount by subtracting the reference feature amount from each of the first feature amounts respectively calculated by the potential measurement sections 3J other than the potential measurement section 3J calculating the first feature amount set to be the reference feature amount.

The state estimator 42 estimates a state of the human 2 on the basis of the second feature amount. State information regarding the estimated state of the human 2 is transmitted to the provision section 43 and the storage 44.

### Others

The embodiments of the present technology have been described above. The present technology is not limited to the embodiments described above, and of course various modifications may be made thereto without departing from the scope of the present technology.

Note that the present technology may also take the following configurations.
(1) A biological information acquiring system, including:
   a plurality of potential measurement sections; and
   a second signal processor,
   each of the plurality of potential measurement sections including
      a reference electrode that is arranged on a measurement-target living body,
      a measurement electrode that is arranged on the living body,
      a biological signal generator that generates a biological signal from a difference between a potential of the reference electrode and a potential of the measurement electrode,
      an analog-digital converter that digitally converts the biological signal, and
      a first signal processor that calculates a first feature amount using the digitally converted biological signal,
   the second signal processor calculating a second feature amount used to estimate a state of the living body, using the first feature amounts respectively calculated by the first signal processors respectively included in the plurality of potential measurement sections.
(2) The biological information acquiring system according to (1), further including
   a state estimator that estimates the state of the living body on the basis of the second feature amount.
(3) The biological information acquiring system according to (1) or (2), in which
   the first signal processor included in each of the plurality of potential measurement sections
      calculates a power for each frequency from the digitally converted biological signal, and
      normalizes the power for each frequency using an integrated intensity in a specified frequency interval to calculate the first feature amount for a normalized intensity.
(4) The biological information acquiring system according to any one of (1) to (3), in which
   the second signal processor calculates the second feature amount by performing signal averaging on the first feature amounts respectively calculated by the plurality of potential measurement sections.
(5) The biological information acquiring system according to any one of (1) to (3), in which
   the biological information acquiring system includes the plurality of potential measurement sections of which the number is at least three,
   from among the first feature amounts respectively calculated by the plurality of potential measurement sections, the second signal processor sets the first feature amount exhibiting a smallest value to be a reference feature amount, and
   the second signal processor calculates the second feature amount by performing signal averaging on the reference feature amount and each of the first feature amounts respectively calculated by the potential measurement sections other than the potential measurement section calculating the first feature amount set to be the reference feature amount.
(6) The biological information acquiring system according to any one of (1) to (3), in which
   from among the first feature amounts respectively calculated by the plurality of potential measurement sections, the second signal processor sets the first feature amount exhibiting a smallest value to be a reference feature amount, and
   the second signal processor calculates the second feature amount by subtracting the reference feature amount from each of the first feature amounts respectively calculated by the potential measurement sections other than the potential measurement section calculating the first feature amount set to be the reference feature amount.
(7) The biological information acquiring system according to (1) or (2), in which
   the first signal processor included in each of the plurality of potential measurement sections calculates, as the first feature amount, a power for each frequency from the digitally converted biological signal,
   the second signal processor calculates an intermediate feature amount by performing signal averaging on the powers for each frequency that are respectively calculated by the plurality of potential measurement sections, and
   the second signal processor normalizes the intermediate feature amount using an integrated intensity in a specified frequency interval to calculate the second feature amount for a normalized intensity.
(8) The biological information acquiring system according to any one of (1) to (7), in which
   each of the plurality of potential measurement sections further includes a bias electrode.
(9) A biological information acquiring apparatus, including
   a plurality of potential measurement sections each including
      a reference electrode that is arranged on a measurement-target living body,
      a measurement electrode that is arranged on the living body,
      a biological signal generator that generates a biological signal from a difference between a potential of the reference electrode and a potential of the measurement electrode,
      an analog-digital converter that digitally converts the biological signal, and
      a first signal processor that calculates a first feature amount using the digitally converted biological signal.
(10) The biological information acquiring apparatus according to (9), in which
   each of the plurality of potential measurement sections further includes a communication section that transmits the first feature amount to an external apparatus.
(11) The biological information acquiring apparatus according to (9), further including
   a second signal processor that calculates a second feature amount used to estimate a state of the living body, using the first feature amounts respectively calculated by the first signal processors respectively included in the plurality of potential measurement sections.
(12) The biological information acquiring apparatus according to (11), further including
   a state estimator that estimates the state of the living body on the basis of the second feature amount.
(13) An information processing apparatus, including
   a second signal processor that calculates a second feature amount used to estimate a state of a living body, using first feature amounts respectively calculated by first signal processors that are respectively included in a plurality of potential measurement sections,
   each of the plurality of potential measurement sections including
      a reference electrode that is arranged on the living body corresponding to a measurement target,
      a measurement electrode that is arranged on the living body,
      a biological signal generator that generates a biological signal from a difference between a potential of the reference electrode and a potential of the measurement electrode,
      an analog-digital converter that digitally converts the biological signal, and
      the first signal processor calculating the first feature amount using the digitally converted biological signal.

### REFERENCE SIGNS LIST

1, 1A to 1H, 1J biological information acquiring system
2 human (living body)
3, 3C, 3J potential measurement section
5, 5C, 5J living-body-attachable device (biological information acquiring apparatus)
5D, 5E, 5F living-body-attachable device (biological information acquiring apparatus, information processing apparatus)
6, 6A to 6C, 6J external apparatus (information processing apparatus)
61G, 61H first external apparatus (information processing apparatus)
30 measurement electrode
31 reference electrode
32 bias electrode
33 analog front-end section (biological signal generator)
34 analog-digital converter
35, 35C, 35J first signal processor
41, 41A to 41C, 41J second signal processor
42 state estimator

1. A biological information acquiring system, comprising:
a plurality of potential measurement sections; and
a second signal processor,
each of the plurality of potential measurement sections including
a reference electrode that is arranged on a measurement-target living body,
a measurement electrode that is arranged on the living body,
a biological signal generator that generates a biological signal from a difference between a potential of the reference electrode and a potential of the measurement electrode,
an analog-digital converter that digitally converts the biological signal, and
a first signal processor that calculates a first feature amount using the digitally converted biological signal,
the second signal processor calculating a second feature amount used to estimate a state of the living body, using the first feature amounts respectively calculated by the first signal processors respectively included in the plurality of potential measurement sections.

2. The biological information acquiring system according to claim 1, further comprising
a state estimator that estimates the state of the living body on a basis of the second feature amount.

3. The biological information acquiring system according to claim 1, wherein
the first signal processor included in each of the plurality of potential measurement sections
calculates a power for each frequency from the digitally converted biological signal, and
normalizes the power for each frequency using an integrated intensity in a specified frequency interval to calculate the first feature amount for a normalized intensity.

4. The biological information acquiring system according to claim 1, wherein
the second signal processor calculates the second feature amount by performing signal averaging on the first feature amounts respectively calculated by the plurality of potential measurement sections.

5. The biological information acquiring system according to claim 1, wherein
the biological information acquiring system comprises the plurality of potential measurement sections of which the number is at least three,
from among the first feature amounts respectively calculated by the plurality of potential measurement sections, the second signal processor sets the first feature amount exhibiting a smallest value to be a reference feature amount, and
the second signal processor calculates the second feature amount by performing signal averaging on the reference feature amount and each of the first feature amounts respectively calculated by the potential measurement sections other than the potential measurement section calculating the first feature amount set to be the reference feature amount.

6. The biological information acquiring system according to claim 1, wherein
from among the first feature amounts respectively calculated by the plurality of potential measurement sections, the second signal processor sets the first feature amount exhibiting a smallest value to be a reference feature amount, and
the second signal processor calculates the second feature amount by subtracting the reference feature amount from each of the first feature amounts respectively calculated by the potential measurement sections other than the potential measurement section calculating the first feature amount set to be the reference feature amount.

7. The biological information acquiring system according to claim 1, wherein
the first signal processor included in each of the plurality of potential measurement sections calculates, as the first feature amount, a power for each frequency from the digitally converted biological signal,
the second signal processor calculates an intermediate feature amount by performing signal averaging on the powers for each frequency that are respectively calculated by the plurality of potential measurement sections, and
the second signal processor normalizes the intermediate feature amount using an integrated intensity in a specified frequency interval to calculate the second feature amount for a normalized intensity.

8. The biological information acquiring system according to claim 1, wherein
each of the plurality of potential measurement sections further includes a bias electrode.

9. A biological information acquiring apparatus, comprising
a plurality of potential measurement sections each including
a reference electrode that is arranged on a measurement-target living body,
a measurement electrode that is arranged on the living body,
a biological signal generator that generates a biological signal from a difference between a potential of the reference electrode and a potential of the measurement electrode,
an analog-digital converter that digitally converts the biological signal, and
a first signal processor that calculates a first feature amount using the digitally converted biological signal.

10. The biological information acquiring apparatus according to claim 9, wherein
each of the plurality of potential measurement sections further includes a communication section that transmits the first feature amount to an external apparatus.

11. The biological information acquiring apparatus according to claim 9, further comprising
a second signal processor that calculates a second feature amount used to estimate a state of the living body, using the first feature amounts respectively calculated by the first signal processors respectively included in the plurality of potential measurement sections.

12. The biological information acquiring apparatus according to claim 11, further comprising
a state estimator that estimates the state of the living body on a basis of the second feature amount.

13. An information processing apparatus, comprising
a second signal processor that calculates a second feature amount used to estimate a state of a living body, using first feature amounts respectively calculated by first signal processors that are respectively included in a plurality of potential measurement sections,
each of the plurality of potential measurement sections including
a reference electrode that is arranged on the living body corresponding to a measurement target,
a measurement electrode that is arranged on the living body,
a biological signal generator that generates a biological signal from a difference between a potential of the reference electrode and a potential of the measurement electrode,
an analog-digital converter that digitally converts the biological signal, and
the first signal processor calculating the first feature amount using the digitally converted biological signal.
